(19) United States
(12) Patent Application Publication
(10) Pub. No.: US 2002/0107177 A1
(43) Pub. Date: Aug. 8, 2002

(54) Use of factor XIIIa inhibitors to treat atherosclerosis

(75) Inventors: Anne Romanic Arnold, Wynnewood, PA (US); Eliot Howard Ohlstein, Glenmoore, PA (US)

Correspondence Address: GLAXOXMITHKLINE, Corporate Intellectual Property - UW2220, P.O. Box 1539, King of Prussia, PA, 19406-0939, US

(73) Assignee: SmithKline Beecham Corporation

(21) Appl. No.: 10/114,787

(22) Filed: Apr. 3, 2002

Related U.S. Application Data
(63) Continuation of application No. 09/525,723, filed on Mar. 14, 2000.
Application No. 09/525,723 is a continuation of application No. 09/093,256, filed on Jun. 8, 1998, now abandoned.
(60) Provisional application No. 60/049,330, filed on Jun. 11, 1997.

(51) Int. Cl. C12Q 1/37; A61K 38/00

(52) U.S. Cl. 514/2; 435/23

(57) ABSTRACT

A method of treating a mammal, preferably a human, having atherosclerotic disease is provided. The method comprises administering to the mammal an inhibitor of Factor XIIIa which is involved in the formation of atherosclerotic lesions. Methods for identifying Factor XIIIa inhibitors are also provided.

### RELATED APPLICATIONS

This application claims the benefit of U.S. Provisional Application No. 60/049,330, filed Jun. 11, 1997.

### FIELD OF THE INVENTION

The present invention relates to treatment of atherosclerosis.

### BACKGROUND OF THE INVENTION

Lipoprotein(a) (Lp(a)) is a lipoprotein complex which resembles low density lipoprotein (LDL) in its content of cholesterol, phospholipid, and apolipoprotein B-100 (apoB-100). The feature which distinguishes Lp(a) from LDL is the presence of an additional protein known as apolipoprotein(a) (apo(a)), which is bound to apoB-100 by disulfide linkage (Scanu et al., 1991, Ann. Intern. Med. 115:209-218). Lp(a) has been identified as a leading inherited risk factor for atherosclerosis (Scanu, 1991, supra; Lawn, 1992, Sci. Am. 266:54-60). Lp(a) becomes deposited within the blood vessel wall in fibrin clots, and accumulates to form fatty streaks that develop into occlusive atherosclerotic plaques with time (Hajjar et al., 1989, Nature 339:303-305). Individuals who develop occlusive atherosclerosis suffer from coronary heart disease (CHD) and elevated plasma Lp(a) has been shown to be an independent risk factor for the development of premature (CHD) (Bostrom et al., 1996, JAMA 276:544-548). In man, plasma Lp(a) concentrations range from <1 to >100 mg/dL, with the average plasma concentration being about 10 mg/dL and with risk associated with levels>30 mg/dL (Bostrom et al., 1996, supra; Liu et al., 1994, Trends in Cardiovascular Medicine 4:40-44). The deposition of Lp(a) within the fibrin clot is one of the initial events contributing to the development of a fatty streak during atherosclerosis. However, the actual mechanism by which Lp(a) becomes stably incorporated into a fibrin clot was unknown until the present invention.

Factor XIII (EC 2.3.2.13) is a transglutaminase enzyme that catalyzes the final step in the clotting cascade (McDonagh et al., 1995, In: Handin, Lux and Stossel (Eds.) Blood, Principles and Practice of Hematology, pp, 1219-1259; Board et al., 1993, Blood Reviews 7:229-242). This enzyme exists as a zymogen and is present in two molecular forms. The tissue form of Factor XIII, identified with platelets, macrophages and placenta, is a dimer comprised of two identical A chains (A2). The plasma form is a tetramer consisting of two A subunits and two B subunits (A2B2). Activation of Factor XIII to Factor XIIIa is mediated by thrombin and is initiated by proteolytic removal of an amino-terminal propeptide followed by a calcium-dependent conformational change that exposes a cysteine residue in the active site. Activation also leads to dissociation of the A2 and B2 subunits of plasma Factor XIII at which point the tissue and plasma forms of Factor XIIIa are identical.

Factor XIIIa, also referred to as plasma transglutaminase, fibrinoligase and fibrin-stabilizing factor, is a calcium-dependent thiol enzyme which catalyzes the formation of amide bonds between endo-g-glutaminyl and endo-e-lysyl residues of proteins. Factor XIIIa is primarily noted for its participation in the clotting cascade where it covalently crosslinks fibrin monomers and converts soft fibrin clots into hard clots (McDonagh et al., 1995, supra; Board et al., 1993, supra). Factor XIIIa is also known to be capable of crosslinking a2-antiplasmin to fibrin, rendering the clot more resistant to lysis and crosslinking extracellular matrix proteins such as fibronectin, vitronectin, and collagen to fibrin, thereby rendering the clot more resistant to lysis, and crosslinking extracellular matrix proteins such as fibronectin, vitronectin, and collagen to fibrin, thereby anchoring the clot to the blood vessel wall.

The function of Factor XIIIa in crosslinking Lp(a) to matrix proteins as part of the atherogenic process was not known prior to the present invention. The present invention relates to effective preventive measures or treatments for atherosclerosis using compounds that inhibit the participation of Factor XIIIa in the atherogenic process and methods for discovering such compounds.

### SUMMARY OF THE INVENTION

The invention relates to a method of treating a mammal having atherosclerotic disease comprising administering to the mammal a Factor XIIIa inhibitor.

The invention also relates to a method of identifying a Factor XIIIa inhibitor comprising (a) incubating a matrix component, an Lp(a) component, and Factor XIIIa in the presence and absence of a test inhibitor, (b) determining whether complex formation between the Lp(a) component and the matrix component was inhibited by the presence of the test inhibitor, and (c) identifying as a Factor XIIIa inhibitor the test inhibitor that inhibited complex formation.

The invention also relates to a method of identifying a Factor XIIIa inhibitor comprising (a) incubating Factor XIIIa and a first substrate pair comprising an Lp(a) component and a matrix component in the presence or absence of a test inhibitor, and (b) incubating Factor XIIIa and a second substrate pair in the presence or absence of the test inhibitor, wherein the second substrate pair comprises any two components that are Factor XIIIa substrates for complex formation, (c) determining whether inhibition of complex formation between the first substrate pair was greater than inhibition of complex formation between the second substrate pair, and (d) identifying as a Factor XIIIa inhibitor the test inhibitor that provided greater inhibition of complex formation between the first substrate pair than between the second substrate pair.

### BRIEF DESCRIPTION OF THE DRAWING

FIG. 1 is a graph showing Factor XIIIa-mediated crosslinking of Lp(a) with fibrinogen as determined by an ELISA assay. Cross-hatched bars show results in the presence of Factor XIIIa and solid bars show results in the absence of Factor XIIIa. Results are presented as the mean and SEM (n=3).

### DETAILED DESCRIPTION OF THE INVENTION

It has been discovered according to the present invention, that Factor XIIIa crosslinks lipoprotein(a) (Lp(a)) with fibrinogen, the soluble precursor of fibrin. It has also been discovered that Factor XIIIa is present in atherosclerotic lesions. Thus the present invention is based on the discovery that Factor XIIIa contributes to the development of atherosclerotic disease. Without wishing to be bound by any particular theory, the inventors believe that Factor XIIIa-mediated crosslinking of Lp(a) to fibrin effectively increases the local concentration of Lp(a) within a fibrin clot, thereby contributing to the pathogenesis of atherosclerosis through the promotion of an anti-fibrinolytic environment, foam cell formation, the generation of a fatty streak, and an increase in smooth muscle cell content.

The invention relates to a method of treating atherosclerotic disease in a mammal, preferably a human, comprising administering to the mammal or human an inhibitor of Factor XIIIa.

“Treating”, as used herein, means any therapy rendered for the purpose of preventing, alleviating, or ablating the disease, whether or not clinical symptoms of the disease are present. Therefore, as a non-limiting example, the invention includes the treatment of healthy individuals who may be at risk for the disease.

“Atherosclerotic disease”, as used herein, means the development of atherosclerotic lesions on and within blood vessels, wherein the atherosclerotic lesions are a risk factor for any deleterious health condition including, but not limited to, atherosclerosis, myocardial infarction, restenosis, stroke and related diseases or conditions.

“Factor XIIIa inhibitor”, as used herein, means a compound which inhibits the activity of Factor XIIIa in crosslinking Lp(a) to matrix proteins, such as determined, for example, by any one or more of the assays described herein.

Inhibitors of Factor XIIIa include but are not limited to, small non-peptide molecules, peptides or peptide analogs comprising specific portions of Factor XIIIa substrates, peptidometics that mimic specific portions of Factor XIIIa substrates, antibodies or engineered fragments thereof directed against Factor XIII or Factor XIIIa, nucleic acids having a sequence which is antisense to all or a portion of the nucleic acid encoding Factor XIII, compounds which inhibit or prevent the expression or activity of Factor XIIIa, and compounds which inhibit or prevent the activation of Factor XIII to Factor XIIIa.

One embodiment of a Factor XIIIa inhibitor is a “Factor XIIIa Lp(a)-matrix specific inhibitor”, defined herein as an inhibitor which has a greater inhibitory effect on Factor XIIIa mediated crosslinking of Lp(a) to matrix proteins than on other aspects of Factor XIIIa function, such as fibrin-fibrin crosslinking as part of the clotting cascade, or the crosslinking of a2-antiplasmin to fibrin, or the crosslinking of extracellular matrix proteins, such as fibronectin, vitronectin, and collagen to fibrin.

A second embodiment of a Factor XIIIa inhibitor is a “Factor XIIIa Lp(a)-fibrin specific inhibitor”, defined herein as an inhibitor which has a greater inhibitory effect on Factor XIIIa mediated crosslinking of Lp(a) to fibrin than on other aspects of Factor XIIIa function such as the crosslinking of fibrin as part of the clotting cascade, or the crosslinking of a2-antiplasmin to fibrin, or the crosslinking of extracellular matrix proteins such as fibronectin, vitronectin, and collagen to fibrin.

To identify a Factor XIIIa inhibitor, a test compound is assessed for efficacy in inhibiting Factor XIIIa in one or more of the assays described herein or in the experimental examples section, or in any other assay for measurement of Factor XIIIa function. Preferably, an in vitro test is used initially to identify a compound effective in inhibiting Factor XIIIa activity. Such in vitro tests include, but are not limited to, tests which assess the affect of the test compound in inhibiting the ability of Factor XIIIa to catalyze or otherwise affect the formation of a complex between an Lp(a) component and a matrix component. Non-limiting examples of the Lp(a) component are the Lp(a) molecule itself, or any portion thereof that contains the apo(a) portion, or the apo(a) portion itself, or any subunit of the apo(a) portion. Non-limiting examples of the matrix component are fibrin, fibrin components such as fibrinogen or other fibrin subunits, fibronectin, vitronectin, and collagen, or any portions thereof. Additionally, to identify inhibitors which prevent the activation of Factor XIII to Factor XIIIa, test compounds can be assessed for their ability to interfere with the thrombin-mediated conversion of Factor XIII to Factor XIIIa. Useful in vitro assays include tests in which thrombin is incubated with Factor XIII and then the ability of the enzyme to catalyze the formation of a complex between an Lp(a) component and a matrix component is evaluated.

To identify a “Factor XIIIa Lp(a)-matrix specific inhibitor” or a “Factor XIIIa Lp(a)-fibrin specific inhibitor”, an inhibitory test compound is identified as described above. Then, the test compound is assessed to determine its inhibitory effect on Factor XIIIa mediated crosslinking of an Lp(a) component to a matrix component, such as fibrin, relative to its inhibitory effect on crosslinking a second substrate pair, wherein the second substrate pair comprises any two components that are Factor XIIIa substrates for complex formation. Examples of other Factor XIIIa substrate pairs include fibrin-fibrin crosslinking, such as occurs as part of the clotting cascade, or a2-antiplasmin-fibrin crosslinking, or the crosslinking of extracellular matrix proteins such as fibronectin, vitronectin, and collagen to fibrin. For example, in vitro tests could be used to determine whether a specific inhibitory test compound caused a greater inhibitory effect in Factor XIIIa mediated formation of Lp(a)-fibrin complexes than in the Factor XIIIa mediated formation of fibrin-fibrin complexes. In assessing the inhibitory effect of the test compound on the second substrate pair, the components of the second substrate pair may be in the in vivo substrates themselves, or representatives or components thereof. For example, fibrinogen could be used instead of the substrate fibrin. Therefore, for the purposes of this invention, the term “Factor XIIIa substrates for complex formation” is defined to include the actual in vivo substrates, or any representatives or components thereof, including but not limited to fibrin or fibrinogen.

The potential inhibitory test compound may also be assessed for efficacy in inhibiting Factor XIIIa by an in vivo assay. As is apparent from the data provided in the experimental examples section, a mouse animal model for the development of atherosclerosis is available. To assay the effectiveness of the Factor XIIIa inhibitor in preventing, alleviating or ablating atherosclerotic disease, the control and test mice are fed a high fat diet to promote development of the atherosclerosis and the test mice are administered the test inhibitory compound by conventional routes to determine the effect of the compound on the development of the disease. The control and test mice are evaluated for the development of atherosclerotic disease by microscopic examination of the aortic sinus region, for instance, as described in the Examples.

Protocols for treatment of atherosclerotic disease in mammals, including humans, by administration of a Factor XIIIa inhibitor will be apparent to those skilled in the art and will vary depending upon the type of disease and the type and age of the mammal or person. Treatment regimes which are contemplated include a single dose or dosage which is administered hourly, daily, weekly, monthly, or yearly. Dosages may vary from 1-1000 mg/kg of body weight of the inhibitor, and will be in a form suitable for delivery of the compound. The route of administration may also vary depending upon the disorder to be treated.

The invention contemplates administration of a Factor XIIIa inhibitor to humans for the purpose of preventing, alleviating, or ablating atherosclerotic disease. The protocol which is described below for administration of Factor XIIIa inhibitor to a human is provided as an example of how to administer Factor XIIIa to a human. This protocol should not be construed as being the only protocol which can be used, but rather, should be construed merely as an example of the same. Other protocols will become apparent to those skilled in the art when in possession of the instant invention.

The Factor XIIIa inhibitor is prepared for administration by being suspended or dissolved in a pharmaceutically acceptable carrier such as saline, salt solution or other formulations apparent to those skilled in such administration. The compositions of the invention may be administered to a mammal or human in one of the traditional modes (e.g., orally, parenterally, transdermally or transmucosally), in a sustained release formulation using a biodegradable biocompatible polymer, or by on-site delivery using micelles, gels and liposomes, or rectally (e.g., by suppository or enema) or nasally (e.g., by nasal spray). Thus, Factor XIIIa inhibitors may be administered to the mammal or human by any route in order that it eventually reaches the target area in the mammal, i.e., the blood vessels, wherein it exerts its effects. The appropriate pharmaceutically acceptable carrier will be evident to those skilled in the art and will depend upon the route of administration. Essentially, for administration to humans, the Factor XIIIa inhibitor is dissolved in about 1 ml of saline solution or other pharmaceutically acceptable solvent or carrier and doses of 1-1000 mg per kg of body weight are administered orally or parenterally once per day to several times per day.

Factor XIIIa inhibitors may also be formulated so as to target specific cell types. For example, it is now known in the art to encapsulate or otherwise formulate compounds such that they are directed to specific receptors on cells. Such formulations include antibody-tagging formulations, receptor-ligand binding formulations and the like.

The invention is further described in detail by reference to the following examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

The examples described herein provide procedures and results which establish that Factor XIIIa inhibitors are useful for the treatment of atherosclerotic disease since, according to the data provided herein, Factor XIIIa is able to crosslink Lp(a) to fibrinogen and Factor XIIIa protein expression is associated with atherosclerotic lesions.

### EXAMPLE 1

### Factor XIIIa is Capable of Crosslinking Lp(a) with Fibrinogen

To determine if Factor XIIIa crosslinks Lp(a) with fibrinogen, Factor XIIIa was incubated with Lp(a) and fibrinogen in solution and then analyzed for Lp(a)-fibrinogen crosslinking over a time course ranging from 30 minutes to 6 hours. Briefly, purified human fibrinogen (Calbiochem, San Diego, Calif.), at a final concentration of 100 μg/ml, was incubated with purified human Lp(a) (Enzyme Research Laboratories, South Bend, Ind.) at a final concentration of 500 μg/ml, in the presence of purified human Factor XIIIa (Enzyme Research Laboratories, South Bend, Ind.) at 30 U/mi. As controls, Factor XIIIa was either omitted from the reaction or 10 mM EDTA was added in the presence of Factor XIIIa to inhibit Factor XIIIa activity. The reactions were conducted at 37° C. in a buffer of 40 mM Tris. 0.15 M NaCl. 5 mM dithiolthreitol, and 10 mM CaC12, at pH 8.3. Factor XIII was preactivated to Factor XIIIa immediately before each experiment by incubating Factor XIII with thrombin at 3 U/ml in 40 mM Tris and 0.15 M NaCl, at pH 8.3 for 1 hour at room temperature. The activation procedure was stopped by adding hirudin at 100 U/ml (Sigma) to inhibit the thrombin. At the end of each time point, the crosslinking reaction was terminated by adding EDTA to a final concentration 25 mM. Additional studies were conducted to study the degree of crosslinking to fibrinogen when increasing amounts of Lp(a) were added to the reaction. Lp(a) was incubated with fibrinogen in the presence or absence of Factor XIIIa as described above, except that the final concentrations of Lp(a) were adjusted to range from 200-800 μg/ml.

Covalently crosslinked complexes formed between fibrinogen and Lp(a) were isolated by immunoprecipitation. A rabbit anti-human fibrinogen antibody (Calbiochem, La Jolla, Calif.) was added to the reaction mixture to a final concentration of 5 μg/ml and then incubated at 4° C. for 18 hours while rotating. Protein A Sepharose beads (Pharmacia, Piscataway, N.J.) (20 mg/ml) were then added to the samples and incubated for an additional 4 hours at 4° C. while rotating. The Lp(a)-fibrinogen complexes, bound by the beads, were then subjected to a series of washes with 1%, 0.5% and 0.05% Triton X-100 in PBS. The beads were pelleted between each wash by centrifugation at 14,000×g and the final pellet was resuspended in Laemmli buffer containing 62.5 mM Tris, pH 6.8, 2% SDS, 5% glycerol, 0.7 M 2-mercaptoethanol and 0.025% bromophenol blue and then heated at 100° C. for 3 minutes.

To identify the presence of Lp(a) within the immunoprecipitated complexes, samples were analyzed by western blot using an antibody to Lp(a). Briefly, samples (30 μl each) were subjected to electrophoresis through a 4-20% polyacrylamide gradient gel (BioRad) (Laemmli, 1970, Nature 227:680-685) and then transferred to a nitrocellulose membrane. Unoccupied binding sites were blocked overnight at 4° C. with 5% nonfat powdered milk in a 0.1 M Tris-HCI buffer, pH 8.0, containing 1.5 M NaCl and 0.5% Triton X-100 (TBST buffer). A sheep anti-human Lp(a) primary antibody (Enzyme Research Laboratories, South Bend, Ind.), diluted in TBST to 10 μg/ml, was then added to the membrane and allowed to incubate for 1 hour at 25° C. The membrane was washed three times, 20 minutes each, with TBST and then incubated for 30 minutes with a secondary antibody conjugated to horseradish peroxidase (Sigma). The membrane was washed as above and the blot was developed using the enhanced chemiluminescence method (Amersham) according to the manufacturer's instructions.

The results from the Western blots demonstrated that, in the presence of Factor XIIIa, increasing amounts of Lp(a) became crosslinked with fibrinogen over time. When Factor XIIIa was omitted from the reaction, Lp(a) was not present in the immunoprecipitated material. Also, when EDTA was added to the reaction to inhibit Factor XIIIa, Lp(a) was not incorporated in the immunoprecipitated material. The results also demonstrated that Lp(a) became crosslinked to fibrinogen in a concentration-dependent manner. When Factor XIIIa was not included in the reaction, Lp(a) was not detected. These results indicate that Factor XIIIa mediates crosslinking between Lp(a) and fibrinogen.

Factor XIIIa-mediated crosslinking of Lp(a) to fibrinogen was also analyzed by an ELISA-based assay. Microtiter ELISA plates (Corning) were coated with purified human fibrinogen (American Diagnostica, Greenwich, Conn.) at a concentration of 80 μg/ml, 100 μl per well, for 40 minutes at room temperature. Unoccupied binding sites were then blocked for 1 hour with 1% bovine serum albumin in Buffer A (40 mM Tris and 0.15 M NaCl, at pH 8.3). Lp(a) (Sigma) was added to the wells to a final concentration of 250, 375 or 500 μg/ml in Buffer A containing 10 mM CaC12 and 5 mM dithiolthreitol. Factor XIIIa (Enzyme Research Laboratories, South Bend, Ind.), preactivated with thrombin as described above, was then added to the wells for a final activity of 30 U/ml. The reactions were allowed to proceed for 2 hours at room temperature and then were stopped by the addition of EDTA to a final concentration of 15 mM. The wells were then washed four times with ELISA wash solution (Kirkegaard and Perry, Gaithersburg, Md.).

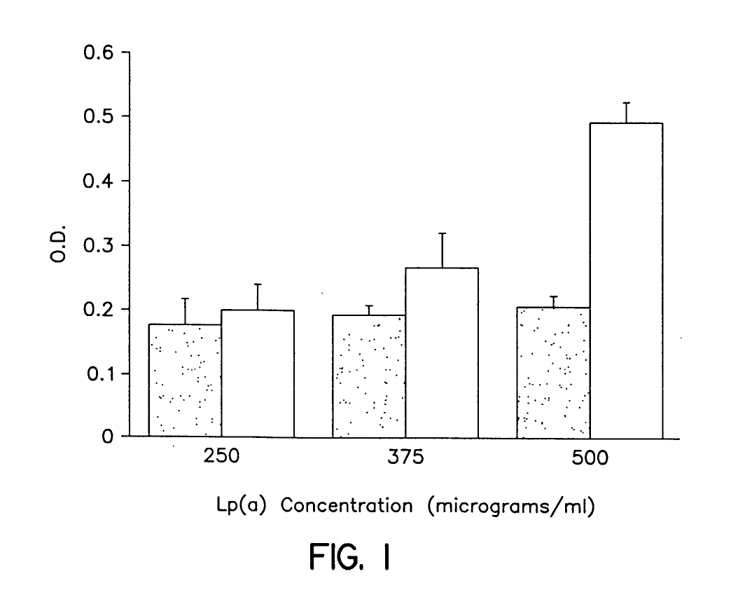

To determine if Factor XIIIa had crosslinked Lp(a) to the immobilized fibrinogen, a sheep anti-Lp(a) antibody (Enzyme Research Laboratories, South Bend, Ind.), diluted to 10 μg/ml in ELISA wash solution, was added to the wells and allowed to incubate for 18 hours at 4° C. The wells were washed as above and then a biotinylated anti-sheep IgG antibody (Vector Laboratories, Burlingame, Calif.), diluted 1:150 in ELISA wash solution, was added and allowed to incubate for 1 hour at room temperature. The wells were washed as above after which 150 μl of streptavidin-β-galactosidase (Gibco/BRL), diluted 1:1,000 in ELISA wash solution, were added for 30 minutes at room temperature. The wells were washed one time with ELISA wash solution and then 150 μl of r-nitrophenyl-β-D-galactopyranoside (Sigma) at 1 mg/ml was added. After 30 minutes of incubation at room temperature the reaction was stopped and the resulting colored product was enhanced by adding 20 μl of 1 N NaOH to each well. Absorbance was then read at a wavelength of 405 nm using a SPECTRAmaxÔ250 Microplate Spectrophotometer (Molecular Devices, Sunnyvale, Calif.). The results demonstrated that, during a 2-hour incubation period in the presence of Factor XIIIa, Lp(a) became crosslinked to the fibrinogen immobilized on the plate in a concentration-dependent manner (FIG. 1). In the absence of Factor XIIIa, a measurable amount of Lp(a) nonspecifically adhered to the fibrinogen-coated plate (FIG. 1).

### EXAMPLE 2

### Factor XIIIa and Lp(a) Expression Co-localize in Human Atherosclerotic Lesions

The expression of Factor XIIIa and Lp(a) in human atherosclerotic lesions was examined by immunohistochemistry. Tissue sections of human coronary arteries with moderate to advanced atherosclerotic lesions were removed and fixed with 10% (wt/vol) phosphate-buffered formalin (Baxter Scientific Products). Following standard histological processing and embedding in paraffin, 6 μm-thick sections were prepared for immunoperoxidase staining using the Vectastain ABC kit (Vector Laboratories, Burlingame, Calif.) according to the manufacturer's instructions. Briefly, endogenous peroxidase was quenched with 0.3% H202 in methanol for 30 minutes. Nonspecific immunoglobulin binding sites were blocked with normal rabbit serum for 1 hour and then the sections were incubated with a sheep anti-Factor XIII primary antibody (2.5 μg/ml, Enzyme Research Laboratories, South Bend, Ind.) or a sheep anti-Lp(a) primary antibody (2.5 μg/ml, Enzyme Research Laboratories, South Bend, Ind.) for 1 hour at room temperature. As a control, serial sections were incubated with sheep IgG (Sigma) instead of the primary antibody. The sections were then incubated for 30 minutes with a biotinylated rabbit anti-sheep IgG secondary antibody (1:200, Vector Laboratories) followed by 30 minutes of incubation with the Vectastain Elite ABC reagent solution. Immunoglobulin complexes were visualized upon incubation with 3,3′-diaminobenzidine (DAB, Vector Laboratories) at 0.5 mg/ml in 50 mM Tris-HCl, pH 7.4 and 3% H202. Sections were washed, counterstained with Gill's Hematoxylin, cleared, mounted with Aquamount (Polysciences), and then examined by light microscopy. Photographic image results showed that Factor XIIIa and Lp(a) are indeed co-expressed within the atherosclerotic lesions. Serial sections that were incubated with sheep IgG instead of Factor XIII or Lp(a) primary antibodies were negative.

### Example 3

### Factor XIIIa is Present in Atherosclerotic Lesions in Mice

Immunohistochemical studies were performed to analyze Factor XIIIa expression in atherosclerotic lesions generated in a mouse model for atherosclerosis. Nine week old C57BL/6J female mice were purchased from the Jackson Laboratory (Bar Harbor, Me.) and allowed a five day acclimatization period. Animals were housed 10 per box in polycarbonate caging with an automatic water system and maintained in a temperature-controlled room with a 12-hour light/dark cycle. The mice were fed either a standard rodent chow containing 4% fat (Purina Mouse Chow 5001, Richmond, Ind.) or a high-fat, high-cholesterol atherogenic diet (Harland Teklad 88051, Madison, Wis.) containing 15% (w/wt) fat, 1.25% cholesterol, and 0.5% cholic acid. At 30 weeks of age (21 weeks of high-fat diet) the mice were sacrificed by cervical dislocation. Blood was collected from the caudal vena cava into tubes containing sodium citrate for Factor XIIIa analysis in plasma as described below. The heart and proximal aorta were excised and washed in phosphate buffered saline to remove blood, and placed in 10% buffered formalin. The basal portion of the heart and proximal aorta were embedded in paraffin and serial sections (20 μm thick) were taken from the appearance to the disappearance of the aortic valves (the aortic sinus region). Alternating sections from each heart were either stained with hematoxylin and eosin, and examined for the presence of atherosclerotic lesions, or left unstained for immunohistochemistry as described below.

Consistent with other published results, (Qiao et al., 1994, Arteriosclerosis and Thrombosis 14(9):1480-1497), lipid-containing lesions were present in the aortic sinus region in 100% of mice fed the atherogenic diet. Lesions were most prominent across the base of the aortic valve sinus and were contiguous with the valve leaflet attachment sites. The tunica intima was thickened with foam cells and myxomatous material. Some lesions contained small foci of mineralization and occasional segmented leukocytes within the foamy material. No lesions were present in mice that were fed standard rodent chow, in either the same or other regions of the aorta.

Heart tissue sections from control mice and from mice fed the high fat diet were prepared for immunoperoxidase staining using the Vectastain ABC kit (Vector Laboratories, Burlingame, Calif.) according to the manufacturer's instructions. Briefly, endogenous peroxidase was quenched with 0.3% H202 in methanol for 30 minutes. Nonspecific immunoglobulin binding sites were blocked with normal rabbit serum for 1 hour and then the sections were incubated with a sheep anti-Factor XIII primary antibody (2.5 μg/ml, Enzyme Research Laboratories, South Bend, Ind.) or a sheep anti-Lp(a) primary antibody (2.5 μg/ml, Enzyme Research Laboratories, South Bend, Ind.) for 1 hour at room temperature. As a control, serial sections were incubated with sheep IgG (Sigma) instead of the primary antibody. The sections were then incubated for 30 minutes with a biotinylated rabbit anti-sheep IgG secondary antibody (1:200, Vector Laboratories) followed by 30 minutes of incubation with the Vectastain Elite ABC reagent solution. Immunoglobulin complexes were visualized upon incubation with 3,3′-diaminobenzidine (DAB, Vector Laboratories) at 0.5 mg/ml in 50 mM Tris-HCI, pH 7.4 and 3% H202. Sections were washed, counterstained with Gill's Hematoxylin, cleared, mounted with Aquamount (Polysciences), and then examined by light microscopy. Photographic image results demonstrated that Factor XIIIa protein was detected in foamy lesions in the aortic valve sinus region, including the base of the valve sinus and the valve leaflets. In contrast, only modest amounts of Factor XIIIa protein were detected in corresponding regions in the aorta of mice fed the control diet.

The disclosures of each and every publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of treating a mammal for atherosclerotic disease comprising administering to the mammal a Factor XIIIa inhibitor.

2. A method according to claim 1, wherein the Factor XIIIa inhibitor is a Factor XIII(a) Lp(a)-matrix specific inhibitor.

3. A method according to claim 1, wherein the Factor XIIIa inhibitor is a Factor XIII(a) Lp(a)-fibrin specific inhibitor.

4. A method according to claim 1, wherein the mammal is a human.

5. A method of identifying an inhibitor of Factor XIIIa comprising:
(a) incubating an Lp(a) component, a matrix component, and Factor XIIIa in the presence or absence of a test inhibitor;
(b) determining whether complex formation between the Lp(a) component and the matrix component was inhibited in the presence of the test inhibitor; and
(c) identifying as a Factor XIIIa inhibitor the test inhibitor that inhibited complex formation.

6. A method according to claim 5, wherein the matrix component is selected from the group consisting of fibrin and a fibrin component.

7. A method of identifying a Factor XIIIa inhibitor comprising:
(a) incubating Factor XIIIa and a first substrate pair comprising an Lp(a) component and a matrix component in the presence or absence of a test inhibitor;
(b) incubating Factor XIIIa and a second substrate pair in the presence or absence of the test inhibitor, wherein the second substrate pair comprises any two components that are Factor XIIIa substrates for complex formation,
(c) determining whether inhibition of complex formation between the first substrate pair was greater than inhibition of complex formation between the second substrate pair; and
(d) identifying as a Factor XIIIa inhibitor the test inhibitor that provided greater inhibition of complex formation between the first substrate pair than between the second substrate pair.

8. A method according to claim 7, wherein the matrix component is selected from the group consisting of fibrin and a fibrin component.

9. A method according to claim 7, wherein the second substrate pair comprises a first member selected from the group consisting of fibrin and a fibrin component and a second member selected from the group consisting of fibrin and a fibrin component.
